(19) United States
(12) Patent Application Publication
(10) Pub. No.: US 2024/0108853 A1
(43) Pub. Date: Apr. 4, 2024

(54) MICROFABRICATED INTRAVASCULAR DEVICE FOR ASPIRATION PROCEDURES

(71) Applicant: SCIENTIA VASCULAR, INC., West Valley City, UT (US)

(72) Inventors: Asmita DULAL, Salt Lake City, UT (US); Clark DAVIS, Holladay, UT (US); Kenneth SMITH, Taylorsville, UT (US); Nolan LESUEUR, West Valley City, UT (US); Scott CUNNINGTON, Salt Lake City, UT (US); William ECCLES, Centerville, UT (US)

(21) Appl. No.: 18/374,564

(22) Filed: Sep. 28, 2023

Related U.S. Application Data
(60) Provisional application No. 63/411,505, filed on Sep. 29, 2022.

(51) Int. Cl. A61M 25/00

(52) U.S. Cl. CPC A61M 25/0051; A61M 2207/10

(57) ABSTRACT

Disclosed is an intravascular device for an aspiration procedure that provides desirable pushability, torque-ability, flexibility, and hoop strength characteristics. The intravascular device incorporates a stacked configuration of microfabricated cut patterns that improve the navigability of the intravascular device during vasculature procedures. The intravascular device may also include a channel configured to hold a radiopaque marker to aid in navigation and location of the intravascular device during a procedure.

### CROSS-REFERENCE TO RELATED APPLICATIONS

This application claims priority to and the benefit of U.S. Provisional Patent Application No. 63/411,505, filed on Sep. 29, 2022, the entirety of which is incorporated herein by reference.

### BACKGROUND

### Technical Field

This disclosure relates generally to intravascular devices for navigating a patient's vasculature to reach a target location, an in particular to intravascular devices for an aspiration procedure.

### Related Technology

Intravascular devices such as catheters are frequently utilized in the medical field to perform delicate procedures deep within the human body. Typically, a catheter is inserted into a patient's femoral, radial, carotid, or jugular vessel and navigated through the patient's vasculature to the heart, brain, or other targeted anatomy as required. Often, a guidewire is first routed to the targeted anatomy, and one or more catheters are subsequently passed over the guidewire and routed to the targeted anatomy. Once in place, the catheter can be used to deliver drugs, stents, embolic devices, radiopaque dyes, or other devices or substances for treating the patient in a desired manner. For example, intravascular devices may be catheters that are routed to a desired target anatomy and provide vacuum suction or aspiration.

In many applications, such an intravascular device must be angled through the tortuous bends and curves of a vasculature passageway to arrive at the targeted anatomy. For example, directing a catheter to portions of the neurovasculature requires passage through the internal carotid artery and other tortuous paths. Such an interventional device requires sufficient flexibility, particularly closer to its distal end, to navigate such tortuous pathways.

However, other design aspects must also be considered. For example, a catheter must also be able to provide sufficient torquability (i.e., the ability to transmit torque applied at the proximal end all the way to the distal end), pushability (i.e., the ability to transmit axial push to the distal end rather than bending and binding intermediate portions), and structural integrity for performing intended medical functions.

With respect to torquability, as a greater length of a catheter is passed into and through a vasculature passageway, the amount of frictional surface contact between the catheter and the vasculature tissue increases, hindering easy movement through the vasculature passage. Transmitting torque from the proximal end to the distal end allows the catheter to rotate and overcome the frictional forces so that further advancement and positioning is possible.

In some cases, portions of the device are microfabricated to increase flexibility. For example, a catheter may include an outer elongated tube that includes a series of machine-cut fenestrations near the distal end and sometimes at other locations. The cuts are typically arranged to define a series of axially extending “beams” that connect a series of circumferentially extending “rings.”

While such microfabricating techniques are beneficial for increasing the flexibility of elongated intravascular components, several challenges remain. Most intravascular devices utilize coils at their distal end to impart desired flexibility characteristics and avoid puncturing or damaging the vasculature or target anatomies. While these coils may provide flexibility, they exhibit poor hoop strength (i.e., the ability to maintain an intended cross-sectional, circular shape) and tend to ovalize upon the provision of vacuum suction or aspiration.

Further, such coils tend to collapse and bunch like a “slinky” when pushed, meaning they can be more difficult to effectively navigate through vasculature and small, tortuous spaces. Such coils also tend to be made from stainless steel or other relatively rigid materials that have poor kink resistance, leading to plastically deformation when taking tight turns.

Intravascular devices may also utilize radiopaque markers, placed at the end of the catheter, to identify and locate the distal tip of the catheter while it is in a patient's vasculature. Such radiopaque markers are often placed over the distal end of the catheters. However, these markers are difficult to secure and are often poorly secured to the distal end of the catheters. Thus, these markers are prone to being detached from the distal end of the catheters during a procedure, such as the while a catheter is being removed from the patient's vasculature. The problem of losing the radiopaque marker within the patient's vasculature is particularly pronounced in neurovascular procedures.

There is thus a long felt and ongoing need for improved intravascular devices and methods that enable the manufacture of such devices.

### SUMMARY

Disclosed are intravascular devices, such as catheters and/or aspiration catheters, with microfabricated outer surfaces. The disclosed intravascular devices have high flexibility at their distal end while retaining and maintaining good torque-ability and pushability for effective navigation of, for example, neuro-vasculature. The disclosed intravascular devices also exhibit improved hoop strength and are thus effective for use as aspiration catheters.

In some embodiments, an intravascular device includes an elongated member extending between a proximal end and a distal end along a longitudinal axis, with a lumen extending from the proximal end to the distal end. The elongated includes a microfabricated outer surface that define a plurality of axially extending beams and circumferentially extending rings, where the microfabricated outer surface contributes to a flexibility gradient of the intravascular device. At least one beam includes an interior surface, an exterior surface, and a pair of opposing lateral surfaces, wherein an angle is formed between the interior surface and one or both of the lateral surfaces. The interior surface and exterior surface of the beam each include an arc length, where the arc length of the exterior surface may be equal to or smaller than the arc length of the interior surface. In some embodiments, the intravascular device is an aspiration catheter.

In some embodiments, one or more polymer layers are applied to the inner and/or outer surfaces of the elongated member. In some embodiments, one or more polymer layers of different hardness and/or modulus are applied at different sections of the elongate member to correspond to a flexibility gradient of the underlying sections of the elongated member.

In some embodiments, an intravascular device includes a marker band channel at a distal-most section or tip of the elongated member. The marker band channel is sized to receive a radiopaque marker band. The grooved marker band channel includes ridges at proximal and distal ends of the channel. The proximal and distal ridges keep the radiopaque marker in place during operation of the intravascular device. Additionally, the channel is grooved to a depth such that the radiopaque marker is substantially flush with an outer diameter of the outer surface of the intravascular device.

In some embodiments, the axial length of the channel is approximately 0.01 to 0.03 inches, such as 0.019, 0.02, 0.025 or a length within a range with endpoints selected from any two of the foregoing values. In some embodiments, a thickness of the radiopaque marker is approximately 0.0015 to 0.0025 inches. When the radiopaque marker has been placed and secured in the channel, the radiopaque marker is approximately flush with the outer diameter of the intravascular device. Adhesive may be wicked between the radiopaque marker and the marker band channel to further mechanically secure the radiopaque marker within the channel.

In one embodiment, a method of manufacturing an intravascular device that includes a “one-beam” configuration comprises the steps of: providing a piece of stock material; passing a blade into the stock material at a cut depth to form a first cut in the stock material without passing completely through the stock material, the blade being oriented such that a cutting edge is substantially perpendicular to a longitudinal axis of the stock material; rotating the stock material relative to the blade without longitudinally advancing the stock material relative to the blade; passing the blade into the stock material to form a second cut; rotating the stock material a second time relative to the blade without longitudinally advancing the stock material relative to the blade; passing the blade into the stock material to form a third cut, where the blade is passed into the stock material for the second and third cuts at the same cut depth as the first cut.

In some embodiments, a proximal section of the disclosed intravascular devices includes a hub or handle equipped with vacuum suction/aspiration capabilities. For example, when the disclosed intravascular devices are routed to a target anatomy, a vacuum hose in the hub may be turned on or vacuum may otherwise be applied to the catheter, providing vacuum suction at the target anatomy. Such vacuum suction enables removal of, for example, clots, emboli, and/or other blockages at the target anatomy.

In some embodiments, the outer surface of the microfabricated component of the intravascular device contains a plurality of cuts (or fenestrations). In some embodiments, multiple different cut patterns are arranged to provide a flexibility profile or gradient. The plurality of cut patterns may include a first cut pattern that may be different from a second cut pattern that may be different from a third cut pattern. In some embodiments, the arrangement of cut patterns involve rotation of the second cut pattern relative to the first cut pattern, and rotation of the third cut pattern relative to the second cut pattern, and/or rotation of beams or sets of beams relative to other beams or sets of beams.

In some embodiments, a third cut pattern includes a two-beam arrangement formed via a one-cut-per-beam manufacturing process (i.e., two cuts to form each two-beam section). In some embodiments, a second cut pattern includes a two-beam arrangement formed via a two-cut-per-beam manufacturing process (i.e., four cuts to form each two-beam section). In some embodiments, a first cut pattern includes a one-beam arrangement formed via a three-cut manufacturing process. In some embodiments, the first cut pattern is distal of the second cut pattern, which is distal of the third cut pattern.

The beams of the various arrangements (e.g., one-beam, two-beam, etc.) include interior/inner surfaces having a first arc length (i.e., inner arc length) and exterior/outer surfaces having a second arc length (i.e., outer arch length). For at least some of the beams, a ratio of the first and second arc lengths ranges from approximately 1.2:1 to 6:1, such as 1.5:1, 2:1, 2.5:1, 3:1, 3.5:1, 4:1, 4.5:1, 5:1, 5.5:1, or a ratio within a range having endpoints defined by any two of the foregoing values. The ratio and lengths of the first and second arc lengths may be controlled and/or influenced by the method of manufacturing the intravascular devices.

Unlike conventional catheters, the disclosed intravascular devices do not require distally extending outer coils to provide desired characteristics to the catheter, such as the flexibility characteristics of the catheter. Rather, the desired characteristics (e.g., pushability, torque-ability, and flexibility) and the flexibility gradient are imparted to the elongated member itself via the microfabricated cut patterns and their stacked arrangement along the longitudinal axis of the elongated member. The microfabricated cut patterns may also work in combination with the polymer coatings to provide an effective flexibility profile. Additionally, desired characteristics are imparted via the material(s) used to construct the intravascular devices. For example, in some embodiments, the disclosed intravascular devices are constructed from nitinol and/or another appropriate alloy.

The microfabricated cut patterns combined with the use of nitinol to form the disclosed intravascular devices provide several benefits.

First, the elongated member, with microfabricated cut patterns, imparts greater hoop strength to the intravascular device as compared to a device with a coil as the major distal structural member. Hoop strength refers to the capability of a device to maintain an intended cross-sectional circular shape. High hoop strength indicates a greater capacity for maintaining the intended cross-sectional, circular shape, which is particularly important in aspiration applications. Low or poor hoop strength means the intended cross-sectional circular shape may not be maintained, meaning the cross-sectional shape of the catheter tends to more readily ovalize when subjected to vacuum forces or other deforming forces. Ovalization (e.g., deformation of the circular shape along an axis) changes the functional circumference or diameter of the intravascular device and can present hurdles to effectively removing clots or other blockages. The disclosed intravascular devices provide effective hoop strength and can therefore effectively and safely remove clots, emboli, and other targets from a patient's vasculature. The effective removal is due, at least in part, to the intravascular device's enhanced capacity to maintain the desired cross-sectional shape.

Second, the disclosed intravascular devices with microfabricated cut patterns effectively transmit torque down a length of the intravascular device (i.e., provide good torque-ability) and effectively transmit pushing forces down the length of the catheter (i.e., provide good pushability). The microfabricated cut patterns enable torque-ability and pushability of the elongated member without requiring coils as a major outer structural component, while still maintaining a desired flexibility profile/gradient along the longitudinal axis of the devices. That is, the microfabricated elongated member itself provides sufficient flexibility at the distal end such that there is no need to attach a distally extending coil to the distal end of the elongated member. Instead, the distal end of the elongated member itself represents the distal end of the device.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIGS. 1 and 2 illustrate example embodiments of an intravascular device with a microfabricated component;

FIG. 3 illustrates a distal section of an example intravascular device with a microfabricated component;

FIG. 4 illustrates a close-up view of a distal tip of an example intravascular device;

FIGS. 5A-5C illustrate a typical cutting process for forming a one-beam cut pattern in a piece of stock material and the structure of the resulting beam from the standard cutting process;

FIGS. 6A-6C illustrate an alternative cutting process and the improved structure of a beam that results from the alternative cutting process;

FIGS. 7A-7E illustrate another alternative cutting process for a two-beam cut pattern and the structure of the resulting beam from the alternative cutting process;

FIGS. 8A-8C illustrate another alternative cutting process; and

FIGS. 9A-9B illustrate comparisons of beams resulting from the typical cutting process and an alternative cutting process.

### DETAILED DESCRIPTION

Disclosed are intravascular devices with microfabricated components. In some embodiments, an intravascular device includes an elongated member extending between a proximal end and a distal end along a longitudinal axis with a lumen extending through the elongated member from proximal end to distal end. The elongated member includes a microfabricated component that defines a plurality of axially extending beams and circumferentially extending rings. The beams include an interior surface, an exterior surface, and a pair of lateral surfaces. In some embodiments, the intravascular device is an aspiration catheter. Although some examples described herein include references to a specific type of intravascular device (e.g., an aspiration catheter), it will be understood that the same components, methods, and principles can be applied to other types of intravascular devices, such as other types of catheters and/or guidewires.

In some embodiments, a polymer layer or coating is applied to the inner surface and/or the outer surface of the elongated member. In some embodiments, the disclosed intravascular devices include a marker band channel at a distal-most section of the intravascular device. The marker band channel is sized to receive a radiopaque marker. The grooved marker band channel includes ridges at proximal and distal ends of the channel. The ridges beneficially keep the radiopaque marker in place during operation of the device. Additionally, the channel is grooved such that the radiopaque marker is substantially flush with the outer diameter of the elongated member at adjacent sections of the elongated member.

In one embodiment, a method of manufacturing an intravascular device having a one-beam section comprises the steps of: providing a piece of stock material; passing a blade into the stock material to form a first cut in the stock material without passing completely through the stock material, the blade being oriented such that a cutting edge is substantially perpendicular to a longitudinal axis of the stock material; rotating the stock material relative to the blade without longitudinally advancing the stock material relative to the blade; passing the blade into the stock material to form a second cut; rotating the stock material a second time, relative to the blade, without longitudinally advancing the stock material relative to the blade; and passing the blade into the stock material to form a third cut.

In some embodiments, a proximal section of a disclosed intravascular device includes a hub or handle equipped with vacuum suction capabilities. For example, when a disclosed intravascular device is routed to target anatomy, a vacuum hose in the hub may be turned on or vacuum may otherwise be applied, providing vacuum suction at the target anatomy. Such vacuum suction enables removal of, for example, clots, and/or other blockages at the target anatomy.

In some embodiments, the microfabricated outer surface includes a plurality of cut patterns. In some embodiments, the cut patterns are arranged with a first cut pattern being different from a second cut pattern which is different from a third cut pattern. In some embodiments, a third cut pattern includes a two-beam arrangement formed via a one-cut-per-beam manufacturing process (i.e., two cuts to form the two beams between each set of rings). In some embodiments, a second cut pattern includes a two-beam arrangement formed via a two-cut-per-beam manufacturing process (i.e., four cuts to form the two beams between each set of rings). In some embodiments, a first cut pattern includes a one-beam pattern formed via a three-cut-per-beam manufacturing process (i.e., three cuts to form the single beam between each set of rings).

Each beam in the various arrangements includes an interior surface having a first arc length and an exterior surface having a second arc length, where a ratio of the lengths of the first and second arc lengths (i.e., a ratio of the inner arc length to the outer arc length) ranges from approximately 1.2:1 to 6:1. The ratio and lengths of the first and second arc lengths may be controlled and/or influenced by the method of manufacturing the intravascular devices.

Unlike conventional catheters, the disclosed intravascular devices do not utilize distally extending coils as the major outer structural component to provide desired characteristics to the catheter, such as flexibility. Rather, the desired characteristics (e.g., pushability, torque-ability, and flexibility) are imparted to the elongated member itself via (at least in part) the microfabricated cut patterns and their arrangement along the longitudinal axis of the disclosed intravascular devices. Additionally, desired characteristics may be influenced or imparted via the material used to construct the intravascular devices. For example, in some embodiments, the disclosed intravascular devices are constructed from nitinol and include one or more polymer coatings selected and disposed to contribute to desired flexibility profiles.

### Overview of Intravascular Devices

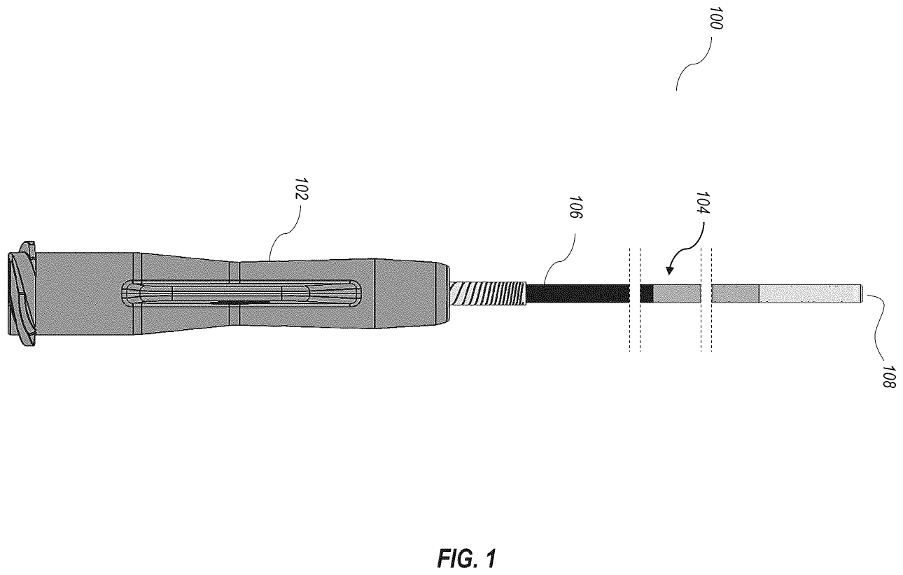

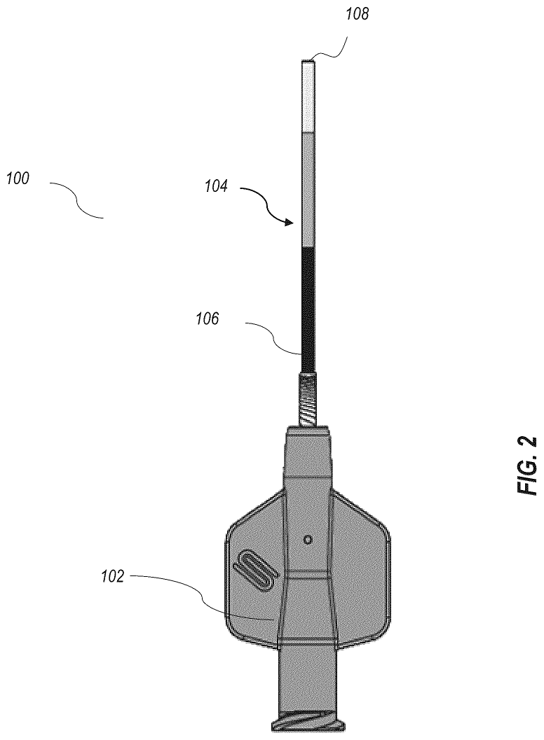

FIGS. 1 and 2 illustrate an exemplary intravascular device 100 that comprises an elongated member 104 extending between a proximal end 106 and a distal end 108. A lumen extends between the proximal end 106 and distal end 108. An inner diameter of the lumen may range from approximately 0.010 inches to 0.050 inches, such as 0.017 inches to 0.04 inches, 0.019, 0.02, 0.025, 0.03, 0.035, 0.037, 0.038, 0.039 inches, or an inner diameter within a range with endpoints defined by any two of the foregoing values. An outer diameter of the intravascular device may range from approximately 0.04 to 0.06 inches, such as 0.045, 0.047, 0.049, 0.051, 0.053, 0.055, 0.057 inches, or an outer diameter within a range with endpoints defined by any two of the foregoing values.

The intravascular device 100 includes an open lumen to enable, for example, aspiration of blood clots or other blockages from a patient's vasculature. An optional handle/hub/torquer 102 may be attached at the proximal end 106. The hub 102 of FIG. 1 is configured as a handle, to be actively grasped by a practitioner; the hub 102 of FIG. 2 is configured as a handle with a paddle, which provides additional stability during use of the intravascular device 100.

The elongated member 104 may be formed from or include a tube structure. The elongated member 104 may include a microfabricated outer surface, which may include a plurality of cut patterns or fenestrations cut into its outer surface. The plurality of fenestrations may be cut into the outer surfaces of the proximal section, the distal section, and/or along the longitudinal axis extending therebetween (i.e., a middle or intermediate section). The fenestrations may be formed by cutting one or more pieces of stock material to form a cut pattern which leaves the fenestrations. The fenestrations can provide a variety of benefits, including increasing the flexibility of the elongated member 104. Further, the fenestrations may be arranged to provide a flexibility gradient along the longitudinal axis of the intravascular devices. In some embodiments, the fenestrations are arranged to provide enhanced flexibility (relative to a similar section of stock material lacking fenestrations) while maintaining sufficient outer circumferential structure for effectively transmitting torque.

The elongated member 104 may be any length necessary for navigating a patient's anatomy to reach a targeted anatomical area. A typical length may be within a range of about 50 to 300 cm, for example. In a catheter embodiment, the outer diameter of the elongated member 104 may be within a range of about 0.020 inches to about 0.350 inches, such as about 0.04 inches to about 0.150 inches, though larger or smaller diameters may also be utilized according to preferences and/or application needs.

In some embodiments, the elongated member 104 includes or is formed from a nickel-titanium alloy having superelastic properties at body temperature. The elongated member 104 may additionally or alternatively be formed from a material having an elastic modulus of about 3000 MPa to about 4500 MPa, or about 3500 MPa to about 4000 MPa. In one embodiment, the elongated member 104 is formed from or includes polyether ether ketone (PEEK). Other polymers with higher moduli may also be utilized where cost and/or fabrication considerations warrant it. The elongated member 104 may additionally or alternatively include stainless steel.

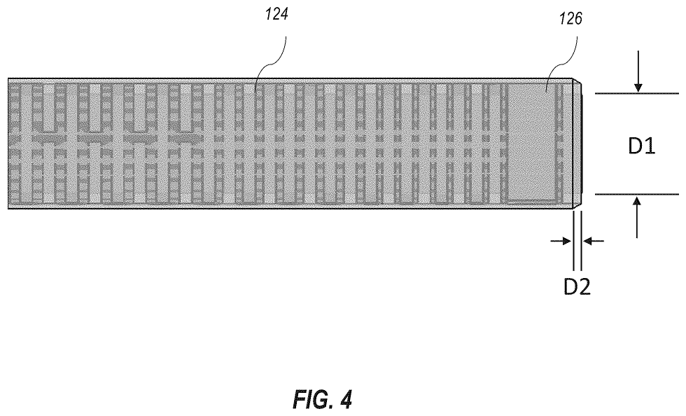

A distal-most section of the disclosed intravascular device includes a marker band channel or groove configured to receive a radiopaque marker and hold the marker in place. The marker band channel is described more fully with respect to FIG. 4.

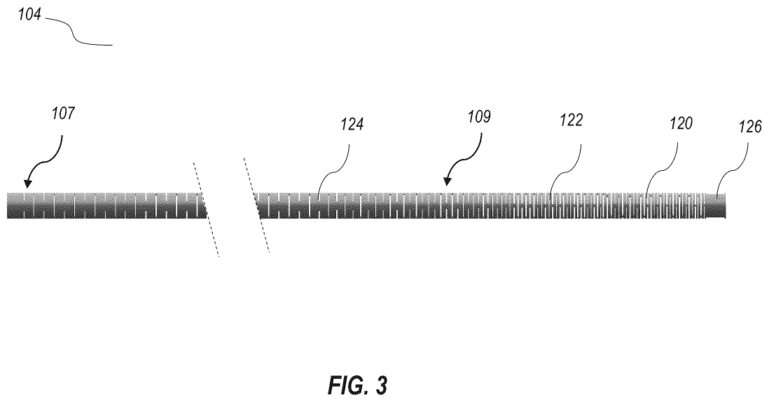

FIG. 3 illustrates a close-up view of the elongated member 104. The break (illustrated by dashed lines) between the proximal section 107 and the distal section 109 indicates a continuous elongated member 104 extending between the proximal section 107 and distal sections 109. The elongated member 104 includes a plurality of different microfabricated cut patterns. The illustrated distal section 109 includes a first microfabricated cut pattern 120, a second microfabricated cut pattern 122, a third microfabricated cut pattern 124, and a marker band channel 126. The proximal section 107 may also include one or more microfabricated cut patterns. For example, the third microfabricated cut pattern 124 may extend to the proximal section 107.

In some embodiments, the third microfabricated cut pattern 124 is a one-cut-per-beam, two-beam section. In some embodiments, the second microfabricated cut pattern 122 is a two-cut-per-beam, two-beam section. In some embodiments, the first microfabricated cut pattern 120 is a three-cut-per-beam, one-beam section. These configurations are described in more detail below. Each section of microfabricated cut patterns transitions into the next section of microfabricated cut patterns; thus, each section of microfabricated cut patterns may include a transition section.

The arrangement of the first, second and third microfabricated cut patterns gives rise to a “stacked” configuration and contributes to or forms a gradient flexibility profile. The gradient provides desired flexibility, pushability, and torque-ability characteristics for efficient and safe operation of the disclosed intravascular devices. For example, the stacked arrangement is configured to impart effective column strength along proximal sections of the intravascular device, while sufficiently reducing the column strength at the distal section. Minimizing the column strength at or near the distal end of the device imparts more flexibility or “give” at the distal end. This flexibility means the intravascular is less likely to puncture tissues or vessels if contacted during navigation to a target anatomy. Rather, the distal end of the intravascular device will tend to buckle or flex when met with resistance from the target anatomy. The distal section 109 can thus safely flex while still maintaining desired shape and diameter during, for example, aspiration procedures.

FIG. 4 illustrates a close-up view of the distal region of the elongated member 104 including the marker band channel 126. The marker band channel 126 is configured to receive a marker, which may be a radiopaque marker to help locate and guide the intravascular device. In some embodiments, the marker is made from tantalum or another appropriate radiopaque material. In some embodiments, an adhesive is applied between the marker and the marker band channel 126 to keep the marker in place within the channel 126. In some embodiments, the marker band channel 126 includes a proximal ridge at a proximal section of the channel 126 and a distal ridge at the most distal section of the channel 126. In some embodiments, the intravascular device 100 extends just beyond the distal ridge. This extension may be chamfered (e.g., at approximately 45°) to aid in navigating through a patient's vasculature atraumatically.

The chamfered extension may have a distance or extension, D2, of approximately 0.001 to 0.0035 inches. In some embodiments, the chamfered extension is approximately 0.002 inches. The inner lumen may have a diameter, D1, of approximately 0.030 to 0.040 inches. For example, in some embodiments, the inner lumen may have a diameter D1 of approximately 0.038 inches. The elongated member 104 may have a wall thickness (including liner) of approximately 0.0030 to 0.0050 inches.

Beneficially, the proximal and distal ridges of the channel 126 keep the marker in place during, for example, extraction of the disclosed intravascular device 100 from a patient's vasculature. Thus, the marker resists being pried from the intravascular device 100 and lost within the patient's vasculature. This problem is particularly poignant when navigating the tight vasculature of the brain. The disclosed intravascular device 100 solves this problem by keeping the marker in place and secured within the channel 126. Further, the marker band channel 126 is grooved such that, when the radiopaque marker is placed within or over the channel 126, an outer diameter of the intravascular device 100 is substantially constant. That is, the outer extent of the marker will be substantially flush with the outer surface of the intravascular device. The marker may be substantially C-shaped and clamped around the channel 126. Adhesive or glue may be applied onto and around the marker, such as onto the joint where the two ends of the C meet. The adhesive may wick in between the marker and the channel 126, thereby further securing the marker into the channel.

In some embodiments, the marker band channel 126 is approximately 0.020 inches to 0.045 inches in length, such as about 0.025 inches to 0.040 inches in length, or a length within a range having endpoints defined by any two of the foregoing values.

### Microfabricated Cut Patterns

Disclosed intravascular devices include microfabricated cut patterns. Examples of various microfabricated cut patterns that may be utilized in addition to the embodiments described herein are illustrated and described in U.S. Application Publication Nos. 2020/0345975 and 2018/0177517, the entire contents of each of which are incorporated herein by reference.

In some embodiments, the microfabricated cut patterns include a plurality of beams (the axially extending segments remaining after a cut is made) and rings (the circular, circumferentially extending segments disposed between each set of cuts). The microfabricated cut patterns may include at least a one-beam arrangement and a two-beam arrangement. A one-beam arrangement includes a single beam disposed between each pair of adjacent rings. A two-beam arrangement includes two beams disposed between each pair of adjacent rings. Other embodiments may include sections with other arrangements, such as a three-beam section with three beams between each pair of adjacent rings.

In some embodiments, some beams are rotated relative to one or more other beams, to avoid forming a straight line of beams on one side or the other of the intravascular devices and/or to avoid or minimize preferred bending axes. For example, an angular offset may be applied at each cut location or every few cut locations (e.g., every second, third, etc.) to intentionally rotate the position of the resulting beams and thereby minimize the formation of preferred bending axes.

In some embodiments, the microfabricated cut patterns are arranged to provide enhanced flexibility (relative to a similar section of stock material lacking fenestrations) while maintaining sufficient outer circumferential structure for effectively transmitting torque. For example, starting at the most distal end of the intravascular device, a one-beam arrangement may be proximally followed by a two-beam arrangement, which may be followed by another two-beam arrangement, which may extend to the proximal end of the intravascular device.

In some embodiments, disclosed intravascular devices include a third microfabricated cut pattern in approximately the proximal section, a second cut pattern in an intermediate section between the proximal and distal sections, and a first cut pattern in approximately the distal section. In some embodiments, the third microfabricated cut pattern includes a two-beam arrangement formed via a conventional one-cut-per-beam process. In some embodiments, the second microfabricated cut pattern includes a two-beam arrangement formed via a two-cut-per-beam process (outlined in more detail below). In some embodiments, the first microfabricated cut pattern includes a one-beam arrangement formed via a three-cut-per-beam process (outlined in more detail below).

In some embodiments, the third microfabricated cut pattern extends from the hub or handle (attached to the intravascular device at its proximal-most end) distally along the longitudinal axis approximately 60 cm to 180 cm, such as about 100 cm to 160 cm or a range using any combination of the foregoing as endpoints. In some embodiments, a strain relief attachment is disposed between the distal end of the hub or handle and the proximal end of the proximal section of the intravascular device.

In some embodiments, the second cut pattern extends distally from a distal end of the third cut pattern along the longitudinal axis of the intravascular device. In some embodiments, the second cut pattern extends from the distal end of the third cut pattern to a proximal end of the first cut pattern. In some embodiments, a length of the second cut pattern is approximately 4 cm to 12 cm, or about 5 cm to about 10 cm, or about 6 cm to about 7 cm, or a length within a range defined by any two of the foregoing values. The distal end of each microfabricated cut pattern may include a transition section to smooth out the transition from one microfabricated cut pattern to the next.

In some embodiments, the first cut pattern extends distally from a distal end of the second cut pattern along the longitudinal axis of the intravascular device. In some embodiments, the first cut pattern extends from a distal end of the second cut pattern to a proximal end of the marker band channel (discussed above). In some embodiments, a length of the first cut pattern is approximately 1 cm to 5 cm, or about 1.5 cm to about 4 cm, or about 2 cm to about 3 cm, or a length within a range defined by any two of the foregoing values.

The arrangement of the first, second, and third microfabricated cut patterns gives rise to a “stacked” configuration and contributes to a gradient of cut patterns. The gradient provides desired flexibility, pushability and torque-ability characteristics for efficient and safe operation of the disclosed intravascular devices. For example, the stacked arrangement is configured to impart some column strength along an approximate proximal section of the intravascular device, while minimizing the column strength at the distal section/end. Minimizing the column strength at or near the distal end of the device imparts more flexibility or “give” at the distal end. This flexibility means the intravascular device does not puncture tissues or vessels as it bumps up against them during navigation to a target anatomy. Rather, the distal end of the intravascular device will buckle or flex when met with resistance from the target anatomy.

In some embodiments, the intravascular device includes one or more polymer coatings. The polymer coatings may be applied on the outer surface of the elongated member over the microfabricated cut patterns. Additionally, or alternatively, the polymer coatings may be applied to an inner surface of the intravascular devices under the microfabricated cut patterns.

The intravascular device may include multiple polymer coatings each with different hardness/durometer and/or modulus. The application of these polymer coatings gives rise to a hardness and/or modulus gradient of coatings. In some embodiments, the gradient of coatings may correspond to the gradient of microfabricated cut patterns. Such matching may enhance the desired characteristics of the device and ensure the desired characteristics (e.g., flexibility, pushability, and/or torque-ability) are maintained.

The disclosed intravascular devices (e.g., aspiration catheters) may be constructed from stock materials. Such stock materials may include, for example, suitable medical-grade catheter materials such as polyetheretherketone (PEEK), polyether block amide (PEBA), other polymers, nitinol, stainless steel, radiopaque materials, and/or combinations thereof. In some embodiments, the elongated member is constructed from a stock nitinol material.

In contrast to conventional aspiration catheters, the disclosed intravascular devices do not use coils extending distally from a hypotube, but rather rely on the gradient of microfabricated cut patterns along the longitudinal axis of the intravascular device to provide a balance of strength and flexibility. Where a coil would normally be used to reduce column strength and increase flexibility, such coils have suboptimal hoop strength and are prone to ovalizing upon application of vacuum. In contrast, for example, the first microfabricated cut 124 pattern (which is the most distal cut pattern) and its linear arrangement along the intravascular device is configured to impart effective hoop strength to the intravascular device while also providing sufficient flexibility and avoiding excessive column strength. As the microfabricated cut patterns of the intravascular devices are disposed along the longitudinal axis and result in gradients of strength and flexibility, different sections of the intravascular devices can be “tuned” for a desired strength and/or flexibility profile.

### Cut Pattern Formation

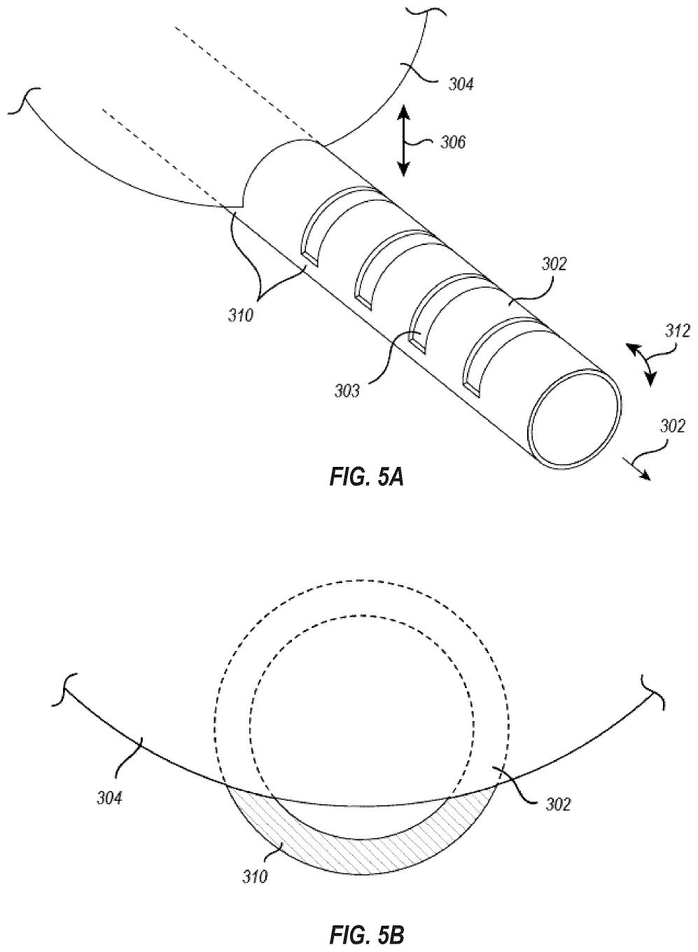

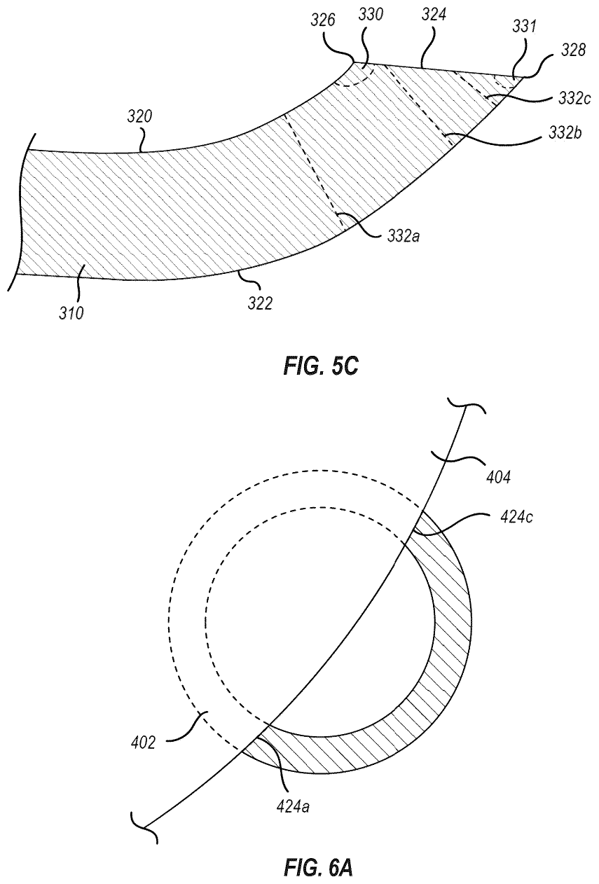

FIGS. 5A-5C illustrate a typical process for forming a one-beam cut pattern in a piece of stock material 302. The stock material 302 (typically a tube structure) is positioned in a cutting machine having a blade 304 (or a plurality of blades). As indicated by arrows 306, the blade 304 is moveable along an axis that is perpendicular to the longitudinal axis of the stock material 302 to form the fenestrations 303. Although the blade 304 is shown here as moving up and down along a vertical axis, other configurations may have a blade (or a plurality of blades) that move along a horizontal axis or even a diagonal axis.

To make a cut, the blade 304 is brought into contact with the stock material 302 and moved inward until the cut is made at the desired depth and a resulting beam 310 remains in the stock material 302. The blade 304 is then withdrawn from the stock material 302. The stock material 302 is then longitudinally moved relative to the blade 304, as indicated by arrow 308, until the next desired cut location is aligned with the blade 304. The process may then be repeated to form the desired number of cuts.

Cut depth and/or spacing between cuts may be varied from one device to the next, or even from one section of a device to another section of the same device. For example, sections intended to form distal portions of an intravascular device may include cuts that are relatively deeper and/or with relatively less spacing in order to increase the relative flexibility at the distal portion.

In some embodiments, the stock material 302 may be rotated between successive cuts or between successive sets of cuts to allow for rotational offsets in the resulting beams, as indicated by arrows 312. Additional details related to cutting machines and related methods of manufacture are described in U.S. Pat. No. 10,232,141, which is incorporated herein by this reference in its entirety.

FIGS. 5B and 5C illustrate in greater detail the structure of the beam 310 that results from the standard cutting procedure shown in FIG. 5A. FIG. 5B shows a front, cross-sectional view of the stock material 302 along a line that runs parallel to the blade path of a particular cut, and FIG. 5C shows an expanded view of an edge section of the resulting beam 310. As shown, the blade 304 typically has a diameter significantly larger than the diameter of the stock material 302 (typical blade diameters may range from 2 to 4 inches, for example). FIG. 5B shows the blade 304 at its deepest point within the stock material 302. After the blade 304 is withdrawn, the resulting beam 310 remains.

As best shown in FIG. 5C, the resulting beam 310 includes an interior surface 320, an exterior surface 322, and two lateral surfaces 324 (only one shown in FIG. 5C). Each lateral surface 324 joins the interior surface 320 along an interior edge 326, and joins the exterior surface 322 along an exterior edge 328. Angle 330 is formed where the interior surface 320 joins the lateral surface 324.

Because of the geometry of the cut, angle 330 is significantly greater than 90 degrees, and will typically be about 135 degrees. As a structural consequence of the size of angle 330, the interior arc length along interior surface 320 is less than the exterior arc length along exterior surface 322. Likewise, while the beam 310 has substantially uniform radial thickness across much of its circumferential length (as indicated by radial line 332a), the radial thickness tapers between the interior edge 326 and the exterior edge 328 (as indicated by progressively shorter radial lines 332b and 332c). Another structural consequence is that edge 328 will be relatively “sharp.” That is, the angle 331 formed between lateral surface 324 and exterior surface 322 will be relatively small, such as about 45 degrees or less.

### Enhanced Cut Pattern Formation

### Two-Cut-Per-Beam Process for a One-Beam Configuration

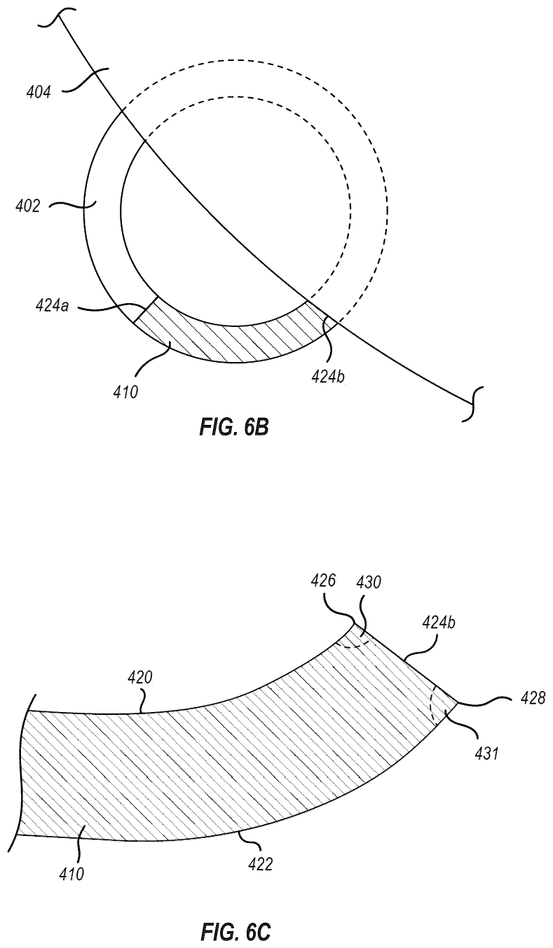

FIGS. 6A through 6B illustrate an alternative method for forming a beam 410 in a section of stock material 402. As shown in FIG. 6A, the blade 404 is first passed into the stock material 402 to a relatively shorter depth compared to the standard cut shown in FIG. 5B. For example, where the standard cut shown in FIG. 5B typically has a depth of about 70% of the stock material diameter or more, the initial cut depth shown in FIG. 6A is approximately 50% (e.g., about 30% to about 70%).

After the initial cut is formed, the stock material 402 is rotated relative to the blade 404 to allow the blade 404 to pass a second time into the stock material 402, as shown in FIG. 6B. The stock material 402 maintains the same longitudinal position relative to the blade during the first and second passes of the blade 404 so that the second cut is within the same plane as the first. During the first cut, a first lateral surface 424a is formed and a temporary lateral surface 424c is formed. The second cut then removes the temporary lateral surface 424c and cuts additional material to form the second lateral surface 424b.

Although the sequence from FIG. 6A to FIG. 6B gives the appearance that the blade 404 is rotated clockwise relative to the stock material 402, it will be understood that this is for illustrative convenience only, and that any suitable means of relative rotation between the stock material 402 and blade 404 may be utilized by rotating the blade 404, the stock material 402, or both. Typically, the stock material 402 will be rotated relative to a rotationally static blade 404. The relative rotation is preferably about 60 degrees (e.g., about 50 degrees to about 70 degrees, or about 55 degrees to about 65 degrees).

FIG. 6C illustrates an expanded view of an edge section of resulting beam 410. The resulting beam 410 includes an interior surface 420, an exterior surface 422, and a pair of lateral surfaces 424 (with the single lateral surface 424b shown here). Each lateral surface 424 joins the interior surface 420 along an interior edge 426 and joins the exterior surface 422 along an exterior edge 428. Angle 430 is formed where the interior surface 420 joins the lateral surface 424.

As compared to angle 330 of the beam 310 shown in FIG. 5C, the angle 430 of beam 410 is markedly smaller. For example, the angle 430 may have a value within a range having a lower endpoint of about 75, 80, 85, or 90 degrees and an upper endpoint of about 130, 120, 110, or 100 degrees. For example, the angle 430 may be approximately 90 degrees such that lateral surface 424b is substantially perpendicular to interior surface 420.

The angle 430 of the beam 410 is also influenced by arc lengths of the interior surface 420 and the exterior surface 422. By comparing FIG. 6C to FIG. 5C, one can see that the ratio of the interior arc length to the exterior arc length is higher in the FIG. 6C embodiment than in the FIG. 5C embodiment, though the exterior arc length remains longer than the interior arc length even in the FIG. 6C embodiment.

The structure of the beam 410 provides a marked improvement over the standard beam 310. For example, the beam 410 avoids the “sharp” exterior edge 428 present in the standard beam 310. In other words, the angle 431 formed between the lateral surface 424b and the exterior surface 422 is greater than 45 degrees, such as about 50 degrees to about 90 degrees.

The dual-pass cutting process has also surprisingly been found to increase manufacturing efficiency and yields as compared to the standard, single-pass process. Even though the number of blade passes is doubled, the dual-pass process requires less depth per cut and typically forms more accurate cuts. This has been found to more than make up for the additional time required to do two cuts per beam. Further, the use of shorter cut depths prolongs the lifespan of the cutting blades.

In some embodiments, the cutting process is governed by the length of each section of microfabricated cut patterns (e.g., the length of the proximal, middle and distal sections) and the degree of cutting distribution. For example, parameters relating to the lengths and degrees may be stored in a software application to be executed by a computer system. Additionally, and/or alternatively, the cutting process may be governed by a desired ratio between the interior and exterior arc lengths.

### Two-Cut-Per-Beam Process for a Two-Beam Configuration

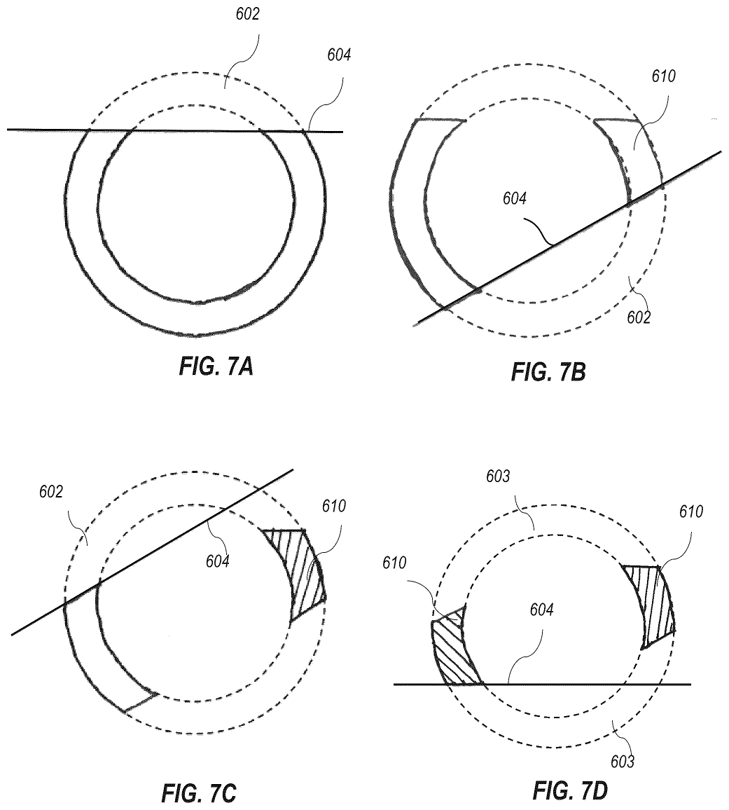

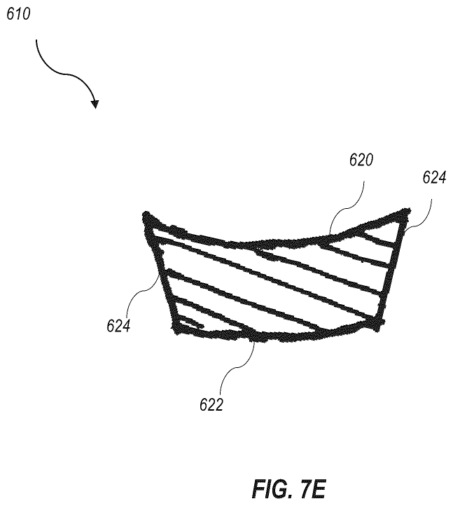

Also illustrated is a four-cut process (two cuts per beam) for forming a two-beam configuration. FIGS. 7A-7D illustrates a four-cut process for forming a two-beam configuration, where each beam is formed from two cuts (sometimes referred to herein as a “two-cut-per-beam, two-beam configuration”). The resulting beams can be substantially the same size and have substantially the same ratio of inner to outer arc lengths.

As illustrated, the blade 604 is first passed into the stock material 602 to a relatively shorter depth compared to the standard cut shown in FIG. 5B. For example, where the standard cut shown in FIG. 5B typically has a depth of about 70% of the stock material diameter or more, the initial cut depth shown in FIG. 7A is approximately 25% (e.g., about 15% to about 35%).

After the initial cut is formed, the stock material 602 is rotated relative to the blade 604 between additional passages of the blade 604 into the stock material 602, as shown in FIGS. 7B-7D. The stock material 602 maintains the same longitudinal position relative to the blade during the first and second passes of the blade 604 so that the second cut is within the same plane as the first. Note that the specific sequence shown in FIGS. 7A-7D is an example only. The relative rotation of the blade 604 and stock material 602 between cuts may follow a different sequence depending on rotation direction, and may instead follow a sequence such as FIG. 7A, FIG. 7C, FIG. 7D, to FIG. 7B, for example.

Although the sequence from FIG. 7A to FIG. 7D gives the appearance that the blade 604 is rotated (e.g., clockwise) relative to the stock material 602, it will be understood that this is for illustrative convenience only, and that any suitable means of relative rotation between the stock material 602 and blade 604 may be utilized by rotating the blade 604, the stock material 602, or both. Typically, the stock material 602 will be rotated relative to a rotationally static blade 604.

As the next pair of beams is formed at the next longitudinal position of the elongated member, the stock material may rotationally offset some amount. For example, the stock material may be rotated 90 degrees relative to the previous pair of beams. This results in each pair of beams being offset by 90 degrees from the previous pair of beams. Beneficially, axially rotating each beam pair or every few beam pairs (e.g., every second, third, etc.) down a length of the stock material can minimize the formation of preferred bending planes.

The process of linearly translating and rotating the stock material to form beams may be repeated as many times as necessary to arrive at a desired length of the two-beam section.

Also beneficially, the disclosed four-cut process produces a more ideal beam cross-sectional shape. As illustrated in FIG. 7E, the resultant beam shape avoids flat and/or sharp artifacts or edges that tend to concentrate stresses and reduce fatigue life of the device. Due to the four-cut process, an outside arc length for the beam (that is, the arc length of the beam's outer surface) will be equal to or even shorter than the interior arc length. Such shapes beneficially reduce “sharp” corners at exterior edges (see, e.g., exterior edge 328 in FIG. 5C), which tend to concentrate mechanical stresses and lower fatigue life of the device. The beam cross-sectional shape as shown in FIG. 7E can also reduce stiffness relative to a similar beam (i.e., a beam with the same cross-sectional area) that has a larger difference between outer arc length and inner arc length.

### Three-Cut-Per-Beam Process for a One-Beam Configuration

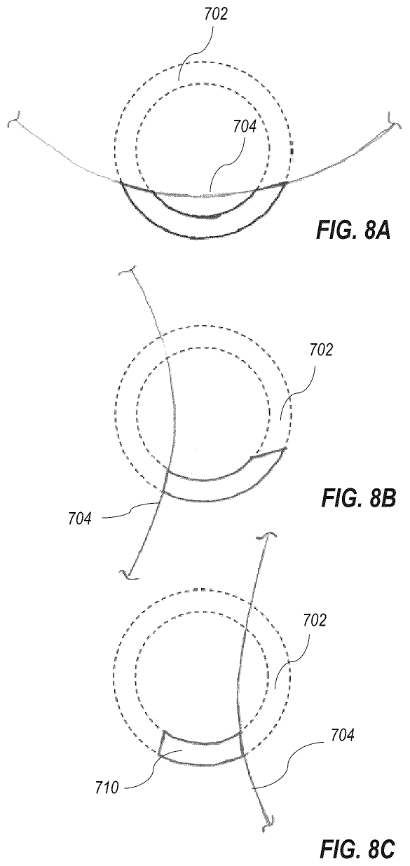

Also illustrated is a three-cut process for forming a one-beam configuration or arrangement. As shown in FIGS. 8A-8C, the blade 704 is first passed into the stock material 702 to a relatively shorter depth compared to the standard cut shown in FIG. 5B. For example, where the standard cut shown in FIG. 5B typically has a depth of about 70% of the stock material diameter or more, the initial cut depth shown in FIG. 8A is approximately 50% (e.g., about 30% to about 70%, typically no greater than about 50% of the diameter of the stock material). Note that the specific sequence shown in FIGS. 8A-8C is an example only. The relative rotation of the blade 704 and stock material 702 between cuts may follow a different sequence depending on rotation direction, and may instead follow a sequence such as FIG. 8B, FIG. 8A, to FIG. 8C, for example.

After the initial cut is formed, the stock material 702 may be rotated relative to the blade 704 to allow the blade 704 to pass a second time into the stock material 702, as shown in FIG. 8B. The stock material 702 maintains the same longitudinal position relative to the blade during the first and second passes of the blade 704 so that the second cut is within the same plane as the first. During the first cut, a first lateral surface and a first temporary lateral surface are formed, similar to the lateral surfaces 424a, 424b illustrated in FIGS. 6A-6B. The second cut then removes the temporary lateral surface and cuts additional material to form a lateral surface of the beam. The stock material 702 may again be rotated and the blade 704 passed a third time into the stock material 702. The third cut removes additional material to form an opposing lateral surface of the resulting beam 710.

Although the sequence from FIG. 8A to FIG. 8C gives the appearance that the blade 704 is rotated clockwise relative to the stock material 702, it will be understood that this is for illustrative convenience only, and that any suitable means of relative rotation between the stock material 702 and blade 704 may be utilized by rotating the blade 704, the stock material 702, or both. Typically, the stock material 702 will be rotated relative to a rotationally static blade 704.

As with other cutting method embodiments described herein, the process of rotating the stock material between cuts, then longitudinally/linearly translating the stock material may be repeated as many times as necessary to arrive at a desired length of the one-beam arrangement. The short relative cut depths used in this method relative to conventional one-cut-per-beam and even two-cut-per-beam one beam sections can prolong the lifespan of the cutting blades. Additionally, the three-cut-per-beam process is particularly suited for larger diameter devices, such as the disclosed aspiration catheter devices having inner diameters of approximately 0.035 inches or other sizes disclosed herein.

Also beneficially, the disclosed three-cut process produces a more ideal beam cross-sectional shape. The resultant beam shape avoids flat and/or sharp artifacts or edges that tend to concentrate stresses and reduce fatigue life of the device. Due to the three-cut process, an exterior/outside arc length (that is, the arc length of the beam's outer surface) will be equal to or shorter than an interior arc length. Such shapes beneficially reduce “sharp” corners at exterior edges (see, e.g., exterior edge 328 in FIG. 5C), which tend to concentrate mechanical stresses and lower fatigue life of the device. The resulting beam cross-sectional shape can also reduce stiffness relative to a similar beam (i.e., a beam with the same cross-sectional area) that has a larger difference between outer arc length and inner arc length.

As discussed above, the beams of any configuration described herein may be rotated from one longitudinal cut location to the next to avoid or minimize alignment of beams along the longitudinal axis in a manner that causes preferred bending planes. For example, lining all the beams of a one-beam section along one side would result in a preferred bending direction along the “spine” of aligned beams. While this may be desirable in some applications, it is typically more preferred that the device bends equally readily in any direction. A “rotational offset” refers to the amount of rotation of the beam or set of beams from one longitudinal position to the next. For example, in a one-beam configuration, a series of cuts may be made at a first longitudinal position to form a first beam at an arbitrary 0° position. The stock material may then be moved linearly/longitudinally relative to the cutting blade to a second longitudinal position. Cuts are then made at the second position to form a second beam. The second beam is preferably rotated relative to the first beam. This process continues as cutting moves longitudinally down the stock material forming successive beams.

It has been surprisingly found that a rotational offset of about 100 degrees to about 150 degrees, in a one-beam configuration, beneficially minimizes the column strength of the corresponding section of the elongate member and thus minimizes the buckling force required to cause buckling under columnar/compressive load. Minimizing buckling force is particularly beneficial at distal sections of the elongate member 104, such as at the section with the first cut pattern 120, to decrease the risk of accidental tissue damage. As an example, the rotational offset may be about 102.5 degrees to about 145 degrees, or about 105 degrees to about 140 degrees, or about 107.5 degrees to about 135 degrees, or about 110 degrees to about 130 degrees, or about 115 degrees to about 125 degrees, or in a most preferred embodiment, about 120 degrees. The rotational offset may be within a range with endpoints defined by any two of the foregoing values.

### Example Benefits of Multi-Pass Cutting Methods

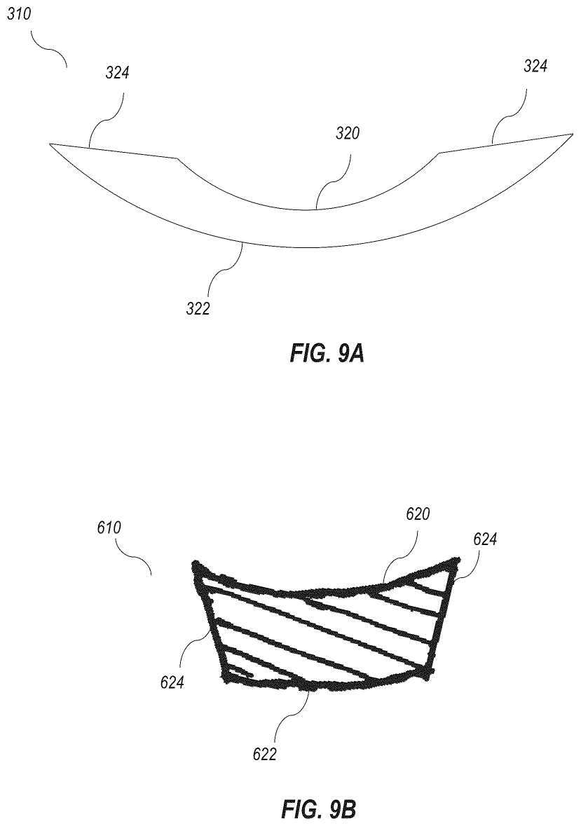

FIGS. 9A and 9B illustrate a comparison of the beams resulting from the process illustrated in FIGS. 5A through 5B and FIGS. 7A through 8C, respectively. FIG. 9A is an illustration of the beam 310 from FIG. 5C, and FIG. 9B is an illustration of the beam 610 from FIG. 7E (and may also be similar to a beam 710 from the process outlined in FIGS. 8A through 8C).

Beam 310 has a pair of lateral surfaces 324, an inner arc length 320 and an outer arc length 322. As can be seen in FIG. 9A, the inner arc length 320 is shorter than the outer arc length 322. Beam 610 has a pair of side surfaces 624, an inner arc length 620 and an outer arc length 622. The inner and outer arc lengths 620, 622 are much closer in length to each other and the inner arc length 620 is even slightly longer than the outer arc length 622. In some embodiments, a ratio of the inner arc length 620 to the outer arc length 622 ranges from approximately 1.2:1 (where the inner arc length 620 is slightly longer than the outer arc length 622) to 6:1 (where the inner arc length 620 is approximately six (6) times longer than the outer arc length 622). In some embodiments, the ratio of the interior arc length to the exterior arc length ranges from approximately 1.2:1 to 6:1, such as 1.5:1, 2:1, 2.5:1, 3:1, 3.5:1, 4:1, 4.5:1, 5:1, 5.5:1 or a ratio within a range having endpoints defined by any two of the foregoing values.

The triple-pass and quadruple-pass cutting processes have also surprisingly been found to increase manufacturing efficiency and yields as compared to the standard, single-pass-per-beam processes. Even though the number of blade passes is doubled or tripled by using the disclosed methods, the multi-pass processes require less depth per cut and typically form more accurate cuts. This has been found, surprisingly, to more than make up for the additional time required to do multiple cuts per beam.

In some embodiments, the cutting process(es) is/are governed by the length of each section of microfabricated cut patterns (e.g., the length of the proximal, middle and distal sections) and the degree of cutting distribution. For example, parameters relating to the lengths and degrees may be stored in a software application to be executed by a computer system. Additionally, and/or alternatively, the cutting process may be governed by a desired ratio between the interior and exterior arc lengths. Further, the cutting process may be governed by where each section of microfabricated cut patterns is placed longitudinally along the elongated member. For example, a microfabricated cut pattern located closer to the proximal end of the elongated member may include a one-cut one-beam process, wherein a microfabricated cut pattern located closer to the distal end of the elongated member may include a three-cut one-beam process.

### Examples

Table 1 provides details regarding outer arc lengths, inner arc lengths and the ratio of the inner to the outer arc length of disclosed beam configurations. Specifically, Table 1 outlines the angle span and cut depth in forming each of the listed cuts. Table 1 also outlines the arc lengths for the inner and outer arcs of each beam and their respective ratios. The minimum and maximum arc lengths are a function of the inner and outer diameters of the stock material used to form the resulting structure. In Table 1, the “3cut-1beam” cut types correspond to the first cut pattern 120, the “2cut-2beam” cut types correspond to the second cut pattern 122, and the “1cut-2beam” cut types correspond to the third cut pattern 124 (see FIG. 3). The “Ratio” is the ratio of the average inner/interior arc length over the average outer/exterior arc length.

TABLE 1

| Cut Type | Avg. Ratio |
|---|---|
| 3cut-1beam Distal | 2.80 |
| 3cut-1beam Proximal | 1.22 |
| 2cut-2beam Distal | 6.19 |
| 2cut-2beam Proximal | 2.30 |
| 1cut-2beam Distal | 1.00 |
| 1cut-2beam Proximal | 1.01 |

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. An intravascular device for an aspiration procedure, the device comprising:
an elongated member including a proximal end, a distal end, and a lumen extending therebetween, the elongated member including a plurality of fenestrations forming a plurality of axially extending beams and a plurality of circumferentially extending rings, each beam including an interior surface, an exterior surface, and two lateral surfaces,
wherein at least one beam has an interior arc length to exterior arc length ratio ranging from about 1.2:1 to about 6:1.

2. The intravascular device of claim 1, wherein the intravascular device is an aspiration catheter.

3. The intravascular device of claim 1, wherein the elongated member includes a one-beam section in which a single beam extends between each pair of adjacent rings.

4. The intravascular device of claim 3, wherein the one-beam section is distal of a two-beam section in which two beams extend between each pair of adjacent rings.

5. The intravascular device of claim 4, wherein the two-beam section comprises a two-cut-per-beam two-beam section.

6. The intravascular device of claim 5, wherein an interior arc length to exterior arc length ratio for each beam of the two-cut-per-beam two-beam section is within a range of about 1.2:1 to about 6:1.

7. The intravascular device of claim 5, wherein the two-beam section further comprises a one-cut-per-beam two-beam section.

8. The intravascular device of claim 7, wherein an interior arc length to exterior arc length ratio for each beam of the one-cut-per-beam two-beam section is less than 1.2.

9. The intravascular device of claim 7, wherein the one-cut-per-beam two-beam section is proximal of the two-cut-per-beam two-beam section.

10. The intravascular device of claim 3, wherein the one-beam section comprises a three-cut-per-beam one-beam section.

11. The intravascular device of claim 10, wherein an interior arc length to exterior arc length ratio for each beam of the three-cut-per-beam one-beam section is within a range of about 1.2:1 to about 6:1.

12. The intravascular device of claim 3, wherein at least some of the beams of the one-beam section are rotated relative to adjacent beams by about 100 degrees to about 150 degrees.

13. The intravascular device of claim 1, further comprising a marker band disposed within a marker band channel, the marker band channel being disposed at a distal tip at or near the distal end of the elongated member.

14. The intravascular device of claim 13, wherein the marker band channel is grooved and has a depth such that an outer surface of the marker band is substantially flush with an outer diameter of the elongated member.

15. The intravascular device of claim 1, further comprising a set of polymer coatings disposed on an inner surface of the elongated member, an outer surface of the elongated member, or both, wherein the set of polymer coatings comprises multiple different polymers, each having a different modulus and/or different hardness, and wherein the multiple different polymers are arranged along the elongated member to contribute to a gradient flexibility profile.

16. The intravascular device of claim 1, wherein the intravascular device omits coils attached to a distal section of the elongated member and extending distally therefrom past the distal end of the elongated member.

17. A method of manufacturing an intravascular device for use in an aspiration procedure, the method comprising:
providing a piece of stock material;
passing a blade into the stock material at a first longitudinal position to form a first cut in the stock material without passing completely through the stock material, the blade being oriented such that a cutting edge is substantially perpendicular to a longitudinal axis of the stock material;
rotating the stock material relative to the blade without longitudinally advancing the stock material relative to the blade;
passing the blade into the stock material to form a second cut;
rotating the stock material a second time relative to the blade without longitudinally advancing the stock material relative to the blade; and
passing the blade into the stock material to form a third cut,
wherein the method results in a single beam between adjacent rings at the first longitudinal position.

18. The method of claim 17, wherein the blade is passed into the stock material at substantially the same depth for the first, second, and third cuts.

19. A method of manufacturing an intravascular device for use in an aspiration procedure, the method comprising:
providing a piece of stock material;
passing a blade into the stock material at a first longitudinal position to form a first cut in the stock material without passing completely through the stock material, the blade being oriented such that a cutting edge is substantially perpendicular to a longitudinal axis of the stock material;
rotating the stock material relative to the blade without longitudinally advancing the stock material relative to the blade;
passing the blade into the stock material to form a second cut;
rotating the stock material a second time relative to the blade without longitudinally advancing the stock material relative to the blade;
passing the blade into the stock material to form a third cut;
rotating the stock material a third time relative to the blade without longitudinally advancing the stock material relative to the blade; and
passing the blade into the stock material to form a fourth cut,
wherein the method results in two beams between adjacent rings at the first longitudinal position.

20. The method of claim 19, wherein the blade is passed into the stock material at the same depth for the first, second, third, and fourth cuts.
